(19) United States
(12) Plant Patent Application Publication
(10) Pub. No.: US 2015/0189822 P1
(43) Pub. Date: Jul. 2, 2015

(54) Salvia plant named 'SER-WISH'

(50) ;

(71) Applicant: John Fisher, Orange (AU)

(72) Inventor: John Fisher, Orange (AU)

(21) Appl. No.: 13/999,016

(22) Filed: Dec. 31, 2013

(51) Int. Cl. A01H 5/00

(52) U.S. Cl. PLT/475

(57) ABSTRACT

A new and distinct Salvia hybrid cultivar named ‘SER-WISH’ is disclosed, characterized by unique bright red-purple to purple flowers and a dark purple calyx color. Plants are upright, dense and have a long flowering season. The new variety is a Salvia, typically produced as an outdoor ornamental plant.

### LATIN NAME OF THE GENUS AND SPECIES

Salvia hybrid

### VARIETY DENOMINATION

‘SER-WISH’

### BACKGROUND OF THE INVENTION

The new cultivar is the product of chance discovery. The new variety originated as a naturally occurring whole plant mutation of the commercial variety of Salvia hybrid ‘Wendy's Wish’ U.S. Plant Pat. No. 21,889.

The new variety was discovered by the inventor, John Fisher, a citizen of Australia, during October of 2010 in a commercial greenhouse in Wonga Park, Australia. After identifying the new variety as a potentially interesting selection, the inventor continued confidential testing and propagation of ‘SER-WISH’ assessing stability of the unique characteristics of this variety.

Asexual reproduction of the new cultivar ‘SER-WISH’ was first performed at a nursery in Wonga Park, Australia, by vegetative cutting in September of 2011. Access to all plants was restricted, as plants were kept in a location that is not open to the public. Through subsequent propagation by vegetative cuttings, six generations have been reproduced, which have shown that the unique features of this cultivar are stable and reproduced true to type.

### SUMMARY OF THE INVENTION

The cultivar ‘SER-WISH’ has not been observed under all possible environmental conditions. The phenotype may vary somewhat with variations in environment such as temperature, day length, and light intensity, without, however, any variance in genotype.

The following traits have been repeatedly observed and are determined to be the unique characteristics of ‘SER-WISH.’ These characteristics in combination distinguish ‘SER-WISH’ as a new and distinct Salvia hybrid cultivar:

1. Bright red-purple to purple flower color.

2. Calyx color dark purple.

3. Upright plant habit

4. Dense, compact plant

5. Long flowering season.

### PARENT COMPARISON

Plants of the new cultivar ‘SER-WISH’ are similar to the parent, Salvia hybrid ‘Wendy's Wish’ in most horticultural characteristics. The new variety, however, produces a different flower color than parent ‘Wendy's Wish’. Additionally ‘SER-WISH’ produces different calyx color than parent ‘Wendy's Wish’.

### COMMERCIAL COMPARISON

Plants of the new cultivar ‘SER-WISH’ are most similar to the parent variety. The inventor is unaware of other commercial varieties to make an effective comparison.

### BRIEF DESCRIPTION OF THE PHOTOGRAPHS

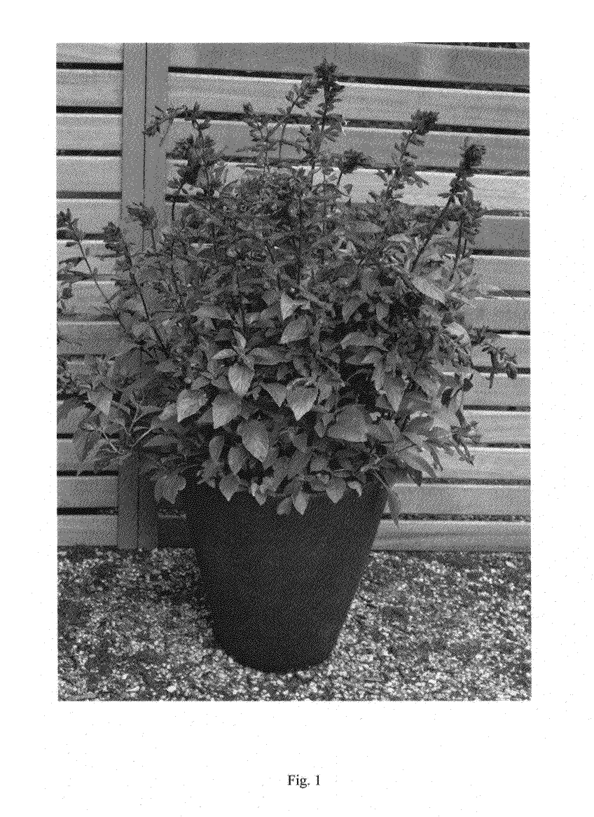

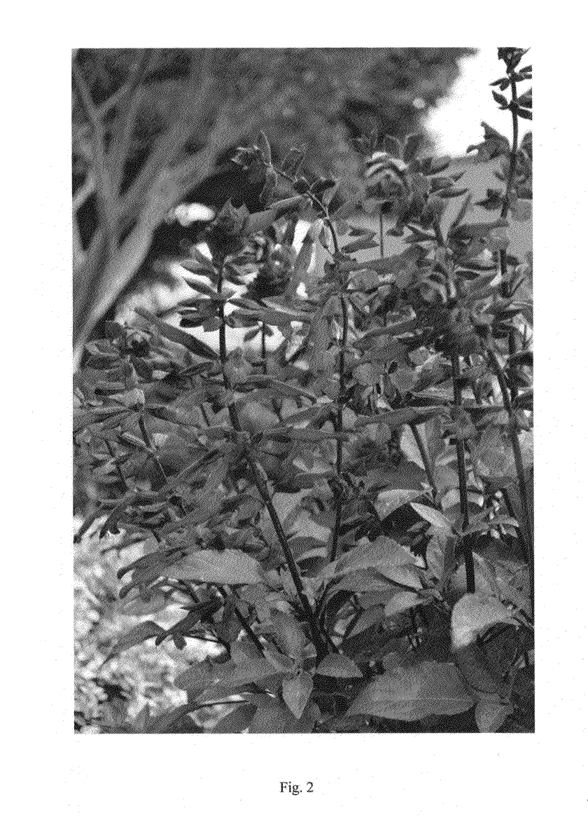

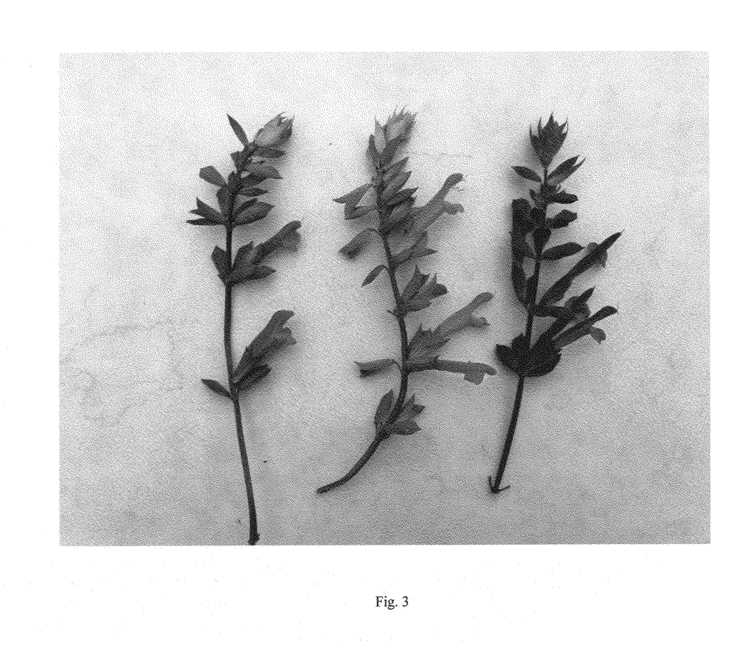

The accompanying photograph in FIG. l illustrates in full color a typical plant of ‘SER-WISH’ grown in a commercial greenhouse in Wonga Park, Australia. This plant is approximately 6 months old, shown planted in a 20 cm. container. FIG. 2 illustrates in full color a close up of the flowers of ‘SER-WISH. FIG. 3 is a comparison of the following cultivars:

Inflorescence on the right is of the new variety, ‘SER-WISH’

Inflorescence in the center is Salvia SAL010-1, application Ser. No. 13/815,340

Inflorescence on the left is the parent variety ‘Wendy's Wish’.

The photographs were taken using conventional techniques and although colors may appear different from actual colors due to light reflectance it is as accurate as possible by conventional photographic techniques.

### DETAILED BOTANICAL DESCRIPTION

In the following description, color references are made to The Royal Horticultural Society Colour Chart 2001, except where general terms of ordinary dictionary significance are used. The following observations and measurements describe ‘SER-WISH’ plants grown ourdoors in Camarillo, Calif. Plants are approximately 16 weeks old. Temperatures ranged from 15° C. to 22° C. day and night. No artificial light, photoperiodic treatments were given to the plants. No chemical treatments were given to plants. Measurements and numerical values represent averages of typical plant types.
Botanical classification: Salvia hybrid ‘SER-WISH’
Propagation:
   Time to initiate roots.—About 10-14 days at approximately 20-21° C.
   Time to produce a rooted cutting.—About 21-28 days at 20-21° C.
Plant:
   Age of plant described.—Approximately 16 weeks from a rooted cutting.
   Height.—Approximately 20 cm.
   Plant spread.—Approximately 17 cm.
   Growth rate.—Moderate to fast.
   Length of primary lateral branches.—Approximately 14 cm.
   Diameter of lateral branches.—Approximately 0.8 cm.
   Quantity of lateral branches.—About 6.
   Stem.—Diameter: Approximately 0.8 cm Color: Near Greyed-Purple group RHS N186C Texture/Pubescence: Glabrous Shape: Quadrangular.
   Internode length.—Approximately 3 cm.
Foliage:
   Leaf.—Arrangement: Opposite Quantity: Approximately 6 to 10 sets of leaves per main branch. Average Length: Approximately 4 cm. Average Width: Approximately 3 cm. Shape of blade: Ovate Apex: Acute Base: Cuneate Attachment: Petiolate Margin: Crenate Texture of top surface: Glabrous Pubescence: Absent Color: Young foliage upper side: Near Yellow-Green Group RHS 146B Young foliage under side: Near Yellow-Green Group RHS 146D Mature foliage upper side: Near Green Group RHS 137A Mature foliage under side: Near Yellow-Green Group RHS 147C Venation: Type: Pinnate Venation color upper side: Near Yellow-Green Group RHS 147C Venation color under side: Near Greyed-Purple group RHS N186D Petiole: Average Length: Approximately 2.5 cm. Diameter: Approximately 3 mm. Color: Near Greyed-Purple group RHS N187A.
Flower:
   Natural flowering season.—Continuously from Mid Spring to Mid Autumn.
   Inflorescence type and habit.—Raceme.
   Flower longevity on plant.—Individual flower approximately 4 to 7 days.
   Quantity of flowers.—About 15 to 20 buds and 3 to 6 fully opened flowers per lateral branch, at one time; or about 10 blooming spikes per plant, for a mature plant of approximately 6 months.
   Inflorescence size.—Diameter: Approximately 4.0 cm. Height: Approximately 22.5 cm.
   Peduncle.—Length: 7.0 cm. Diameter: 0.2 cm. Color: Near Greyed-Purple group RHS N186B Orientation: Erect Strength: Strong.
   Petals.—Petal arrangement: Fused into a corolla tube, emerging with asymmetrical upper and lower lips. Corolla tube color: Near Red-Purple group RHS 64A and Purple N77A Corolla length: 4.5 cm. Corolla width: 1.5 cm. (including lower lip) Corolla Tube surface: Pubescent Corolla lip number: 2 Upper lip shape: 1 hooded orbicular lip. Upper lip color: Near Red-Purple group RHS 64A and Purple N77A Upper lip surfaces: Pubescent Upper lip length: 1.3 cm. Upper lip length: 0.8 cm. Upper lip apex: Obtuse Margin: Entire Corolla lower lip shape: 3 lobed. Lower lip base: Basally fused. Lower lip apex: Obtuse Lower Lip surfaces: Glabrous Corolla lower lip color: upper surface: Near Red-Purple 71B Corolla lower lip color: lower surface: Near Red-Purple 71C Lower lip length: 1.0 cm. Lower Lip width: 1.3 cm. Margin: Entire Petaloids: Not present.
   Bud.—Shape: Oblanceolate Length: 2 cm. Diameter: 0.8cm. Color: Near Red-Purple 72B and Purple N77A Texture: Pubescent.
   Calyx.—Shape: Fused, oblanceolate Length: Approximately 2 cm. Width: Approximately 0.8 cm. Texture: Pubescent Color: Near Purple N77A and Red-Purple 72B.
   Sepals.—Type: Fused Apex: Acute Margin: Entire Texture: Pubescent.
   Bracts.—Shape: Elliptic Apex: Acuminate Margin: Entire Color: Near Purple N77A Texture: Smooth Shape: Concave.
Reproductive organs:
   Stamens.—Number: 2 Filament length: Approximately 2 cm.
   Anthers.—Shape: Elliptical Length: Approximately 3 mm. Color: Near Greyed-Purple group RHS 186B Pollen: Absent.
   Pistil.—Number: 1 Length: Approximately 4.5 cm. Style: Length: Approximately 0.4 cm. Color: Near Red-Purple 59A Stigma: Shape: 2 lobed Color: Near Red-Purple 59A Ovary color: Near Yellow-Green 153D.
Other characteristics:
   Seeds and fruits.—None observed to date.
   Disease/pest resistance.—Typical for Salvia.

What is claimed is:

1. A new and distinct cultivar of Salvia hybrid plant named ‘SER-WISH’ as herein illustrated and described.
